(19) United States
(12) Patent Application Publication
(10) Pub. No.: US 2012/0296241 A1
(43) Pub. Date: Nov. 22, 2012

(54) ULTRASOUND NEUROMODULATION FOR TREATMENT OF AUTISM SPECTRUM DISORDER AND ALZHEIMERS DISEASE AND OTHER DEMENTIAS

(76) Inventor: David J. Mishelevich, Playa del Rey, CA (US)

(21) Appl. No.: 13/475,985

(22) Filed: May 20, 2012

Related U.S. Application Data
(60) Provisional application No. 61/488,754, filed on May 22, 2011.

(51) Int. Cl. A61N 7/00

(52) U.S. Cl. 601/2

(57) ABSTRACT

Disclosed are methods and systems and methods for non-invasive neuromodulation using ultrasound for the Treatment of Autism Spectrum Disorders and Alzheimer's Disease and other dementias. The neuromodulation can produce acute or long-term effects. The latter occur through Long-Term Depression (LTD) and Long-Term Potentiation (LTP) via training. Included is control of direction of the energy emission, intensity, frequency, pulse duration, firing pattern, and phase/intensity relationships to targeting and accomplishing up regulation and/or down regulation.

### CROSS REFERENCE TO RELATED APPLICATIONS

This patent application claims priority to Provisional Patent Application Nos. 61/488,754 filed May 22, 2011, entitled “ULTRASOUND NEUROMODULATION TREATMENT OF AUTISM SPECTRUM DISORDER,” 61/508,612, filed Jul. 16, 2011 and entitled “ULTRASOUND NEUROMODULATION TREATMENT OF ALZHEIMER'S DISEASE.”

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity for modification of neurological function.

### BACKGROUND OF THE INVENTION

It has been demonstrated that focused ultrasound directed at neural structures can stimulate those structures. If neural activity is increased or excited, the neural structure is up regulated; if neural activated is decreased or inhibited, the neural structure is down regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested. The above approach is incorporated in a patent application submitted by Tyler (Tyler, William, James P., PCT/US2009/050560, WO 2010/009141, published 2011 Jan. 21).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. In cases different than treatment of motor disorders, the stimulation frequency for inhibition is 300 Hz or lower (depending on condition and patient). In the case of motor disorders, based on experience with Deep-Brain Stimulation (DBS) with implanted electrodes, treatment for Parkinson's Disease or Essential Tremor would typically be 130 pulse per second and Dystonia in the range of 135-185 pulses per second (all superimposed on the carrier frequency of say 0.5 MHz or similar) although this will be both patient and condition specific. Repeated sessions result in long-term effects. In other situations, high frequencies (defined as being in the range of 500 Hz to 5 MHz) are excitatory and activate neural circuits. This works whether the target is gray or white matter. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

Because of the utility of ultrasound in the neuromodulation of deep-brain structures, it would be both logical and desirable to apply it to the treatment of motor disorders.

### SUMMARY OF THE INVENTION

It is the purpose of this invention to provide methods and systems for non-invasive neuromodulation using ultrasound for modification of cognitive neurological function, including treatment of Autism Spectrum Disorders and Alzheimer's Disease and other dementias. Such neuromodulation can produce acute effects or Long-Term Potentiation (LTP) or Long-Term Depression (LTD). Included is control of direction of the energy emission, intensity, frequency, pulse duration, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

Autism Spectrum Disorders:

Autism Spectrum Disorders include Autism, Asperger's Syndrome, and Atypical Autism. Sometimes Rett Syndrome and Childhood Disintegrative Disorder are included. Multiple targets can be neuromodulated singly or in groups to treat Autism Spectrum Disorders. To accomplish the treatment, in some cases the neural targets will be up regulated and in some cases down regulated, depending on the given neural target. Targets have been identified by such methods as PET imaging, fMRI imaging, and clinical response to Transcranial Magnetic Stimulation (TMS).

For treatment of Autism, primary neural targets are the Parietal Lobe, Amygdala, Anterior Cingulate Gyms, and Caudate Nucleus. The Parietal Lobe was identified by Wong et L. (Wong T K, Fung P C, Chua S E, and G M McAlonan, “Abnormal spatiotemporal processing of emotional facial expressions in childhood autism: dipole source analysis of event-related potentials,” Eur J Neurosci. 2008 July; 28(2):407-16), Gomot et al. (Gomot M, Belmonte M K, Bullmore E T, Bernard F A, and S. Baron-Cohen, “Brain hyper-reactivity to auditory novel targets in children with high-functioning autism, “Brain. 2008 September; 131(Pt 9):2479-88. Epub 2008 July 31) and Shafritz et al. (Shafritz K M, Dichter G S, Baranek G T, and A Belger, “The neural circuitry mediating shifts in behavioral response and cognitive set in autism, “Biol Psychiatry. 2008 May 15; 63(10):974-80. Epub 2007 Oct. 4). The Amygdala was identified by Pinkham et al. (Pinkham A E, Hopfinger J B, Pelphrey K A, Piven J, and D L Penn, “Neural bases for impaired social cognition in schizophrenia and autism spectrum disorders”, Schizophr Res. 2008 February; 99(1-3):164-75. Epub 2007 Nov. 28). The Anterior Cingulate Gyms was identified by Haznedar et al. (Haznedar M M, Buchsbaum M S, Wei T C, H of P R, Cartwright C, Bienstock C A, and E. Hollander, “Limbic circuitry in patients with autism spectrum disorders studied with positron emission tomography and magnetic resonance imaging,” Am J. Psychiatry. 2000 December; 157(12):1994-2001) and the Caudate Nucleus by Degirmenci et al. (Degirmenci B, Miral S, Kaya G C, Iyilikçi L, Arslan G, Baykara A, Evren I, and H. Durak, “Technetium-99m HMPAO brain SPECT in autistic children and their families,” Psychiatry Res. 2008 Apr. 15; 162(3):236-43. Epub 2008 Mar. 4). A subset of these targets would also work and other targets may be discovered as well.

In the application of the therapeutic ultrasound, the Parietal Lobe would be down regulated, and the Anterior Cingulate Cortex (ACC), the Amygdala, and Caudate Nucleus up regulated.

Alzheimer's Disease:

Multiple targets can be neuromodulated singly or in groups to treat Alzheimer's Disease and other dementias. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). For treatment of Alzheimer's Disease and other dementias, primary neural targets are the Hippocampus (Henneman W J, Sluimer J D, Barnes J, van der Flier W M, Sluimer I C, Fox N C, Scheltens P, Vrenken H, and F Barkhof, “Hippocampal atrophy rates in Alzheimer disease: added value over whole brain volume measures,” Neurology. 2009 Mar. 17; 72(11):999-1007), Posterior Cingulate Gyms (PCG)(Awad M, Warren J E, Scott S K, Turkheimer F E, and R J Wise R J, “A common system for the comprehension and production of narrative speech,” J Neurosci. 2007 Oct. 24; 27(43):11455-64), Temporal Lobe (Zhang Y, Londos E, Minthon L, Wattmo C, Liu H, Aspelin P, and L O Wahlund, “Usefulness of computed tomography linear measurements in diagnosing Alzheimer's disease,” Acta Radiol. 2008 February; 49(1):91-7), Formix (Ringman J M, O'Neill J, Geschwind D, Medina L, Apostolova L G, Rodriguez Y, Schaffer B, Varpetian A, Tseng B, Ortiz F, Fitten J, Cummings J L, and G Bartzokis G, “Diffusion tensor imaging in preclinical and presymptomatic carriers of familial Alzheimer's disease mutations,” Brain. 2007 July; 130(Pt 7):1767-76. Epub 2007 May 23), Mamillary Body (Copenhaver B R, Rabin L A, Saykin A J, Roth R M, Wishart H A, Flashman L A, Santulli R B, McHugh T L, and A C Mamourian, “The formix and mammillary bodies in older adults with Alzheimer's disease, mild cognitive impairment, and cognitive complaints: a volumetric MRI study,” Psychiatry Res. 2006 Oct. 30; 147(2-3):93-103. Epub 2006 Aug. 22), and Dentate Gyms (Bramham C R, “Control of synaptic consolidation in the dentate gyms: mechanisms, functions, and therapeutic implications,” Prog Brain Res. 2007; 163:453-71), all of which are to be up regulated. An example of a non-Alzheimer's dementia is TemporalFrontal dementia related to the Anterior Cingulate and the Frontoinsular cortex (Seeley, W., Carlin, Danielle A., Allman, J, Macedo, M., Bush, Clarissa, Miller, B. and S. J. DeArmond, “Early Frontotemporal Dementia Targets Neurons Unique to Apes and Humans,” Ann Neurol 2006; 60:660-667; Published online Dec. 22, 2006 in Wiley InterScience, (www.interscience.wiley.com). DOI: 10.1002/ana.21055).

In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging. Tyler (PCT/US2009/050560, WO 2010/009141) lists motor disorders as a potential area of treatment, but does not pass the test of embodiment because neither the relevant targets nor approaches are described.

While ultrasound can be focused down to a diameter on the order of one to a few millimeters (depending on the frequency), whether such a tight focus is required depends on the conformation of the neural target.

### BRIEF DESCRIPTION OF THE DRAWINGS

FIG. 1 shows a block diagram of the control circuit.

FIG. 2 shows targeting of the Anterior Cingulate Gyms, Caudate Nucleus, Parietal Lobe, and Amygdala for the treatment of Autism Spectrum Disorder.

FIG. 3 shows ultrasonic-transducer targeting of the Hippocampus, Formix, Mamillary Body and Dentate Gyms, Posterior Cingulate Gyms (PCG), and Temporal Lobe for the treatment of Alzheimer's Disease and other dementias.

### DETAILED DESCRIPTION OF THE INVENTION

It is the purpose of this invention to provide methods and systems for non-invasive deep-brain neuromodulation using ultrasound for the treatment of Autism Spectrum Disorders and Alzheimer's Disease and other dementias. Such neuromodulation systems can produce applicable acute or long-term effects. The latter occur through Long-Term Depression (LTD) or Long-Term Potentiation (LTP) via training. Included is control of direction of the energy emission, intensity, frequency, pulse duration, firing pattern, and phase/intensity relationships to targeting and accomplishing up-regulation and/or down-regulation.

The stimulation frequency for inhibition is 300 to 500 Hz or lower (depending on condition and patient). In one embodiment, the modulation frequency of lower than approximately 500 Hz is divided into pulses 0.1 to 20 msec. repeated at frequencies of 2 Hz or lower for down regulation. The stimulation frequency for excitation is in the range of 500 Hz to 5 MHz. In one embodiment, the modulation frequency of higher than approximately 500 Hz. is divided into pulses 0.1 to 20 msec. repeated at frequencies higher than 2 Hz for up regulation. The stimulation frequency for excitation (carrier frequency on which the lower-rate stimulation frequency is superimposed) is in the range of 500 Hz to 5 MHz. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is modulated at the lower rate to impact the neuronal structures as desired (e.g., say 300 Hz or lower for inhibition (down-regulation) or 500 Hz or higher for excitation (up-regulation). A wide range in quantity of ultrasound transducers can be used, but typically will fall in the range of one to ten. Ultrasound therapy can be combined with therapy using other devices (e.g., Transcranial Magnetic Stimulation (TMS)). In some embodiments the modulation is pulsed with the pulses up to 2 msec. in length and repeated at 2 Hz or shorter for inhibition and higher for excitation. In other embodiments, mechanical perturbations are applied to the ultrasound transducers to move them radially or axially.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon or Blatek in the U.S. are another custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

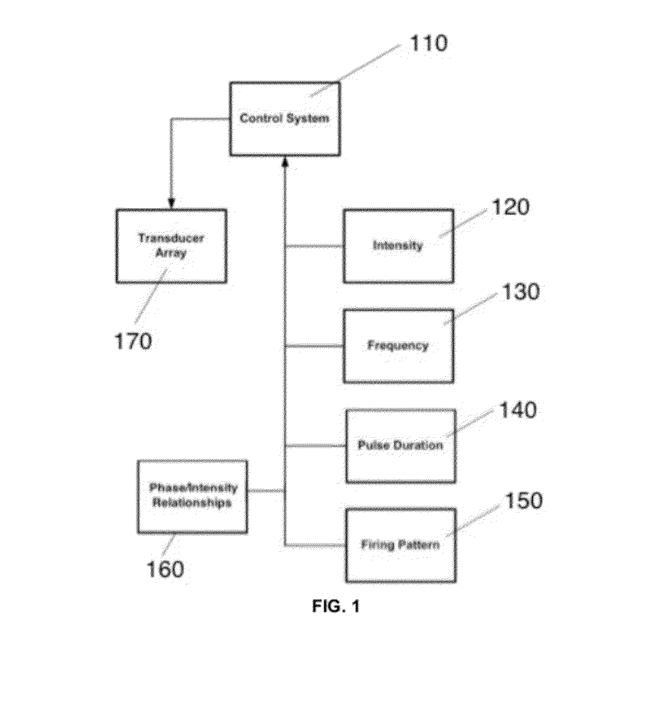

FIG. 1 shows an embodiment of a control circuit for ultrasound neuromodulation variables. The positioning and emission characteristics of transducer array 170 are controlled by control system 110 with control input with neuromodulation characteristics determined by settings of intensity 120, frequency 130, pulse duration 140, firing pattern 150, and phase/intensity relationships 160 for beam steering and focusing on neural targets. In another embodiment multiple ultrasound transducers whose beams intersect at that target replace an individual ultrasound transducer for a given target.

Autism Spectrum Disorders:

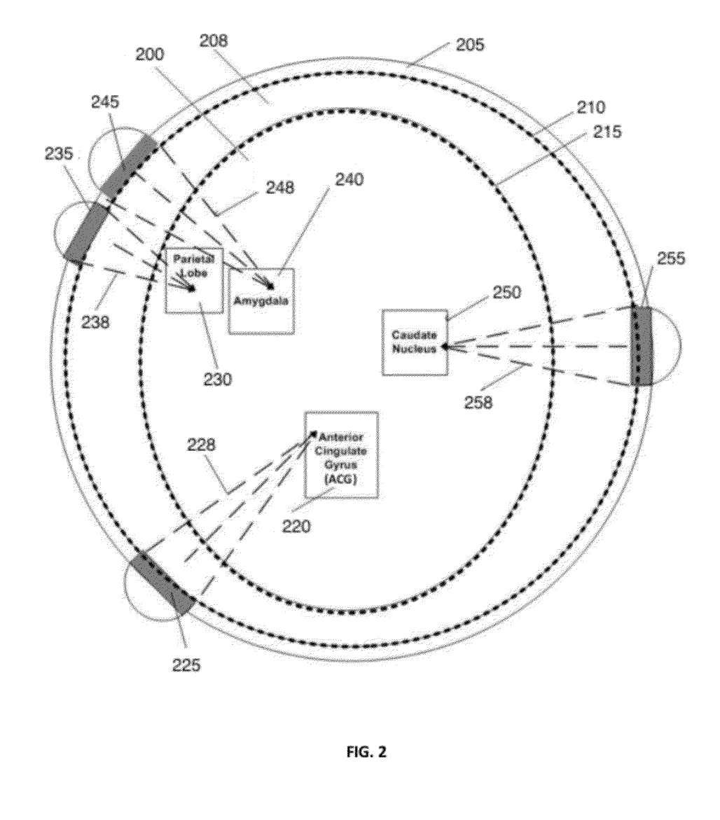

FIG. 2 shows a set of ultrasound transducers targeting to treat Autism Spectrum Disorders. The head 200 contains the four targets, Anterior Cingulate Gyms (ACG) 220, Parietal Lobe 230, Amygdala 240, and Caudate Nucleus 250. Note that while these four targets are covered here, fewer can work as well, or an addition or substitution of other targets identified in the future. These targets are hit by ultrasound from transducers 225, 235, 245, 255, fixed to track 205. Ultrasound transducer 225 with its beam 228 is shown targeting the Anterior Cingulate Gyms (ACG) 220 which would be up regulated, transducer 235 with its beam 238 is shown targeting the Parietal Lobe 230 which would be down regulated, transducer 245 with its beam 248 is shown targeting the Amygdala 240 which would be up regulated, and transducer 255 with its beam 258 is shown targeting the Caudate Nucleus 250 which would be up regulated. For ultrasound to be effectively transmitted to and through the skull and to brain targets, coupling must be put into place. Ultrasound transmission (for example Dermasol from California Medical Innovations) medium 208 is interposed with one mechanical interface to the frame 205 and ultrasound transducers 225, 235, 245, and 255 (completed by a layer of ultrasound transmission gel 210) and the other mechanical interface to the head 200 (completed by a layer of ultrasound transmission gel 215). In another embodiment the ultrasound transmission gel is only placed at the particular places where the ultrasonic beams from the transducers are located rather than around the entire frame and entire head. In another embodiment multiple ultrasound transducers whose beams intersect at that target replace an individual ultrasound transducer for a given target.

Alzheimer's Disease:

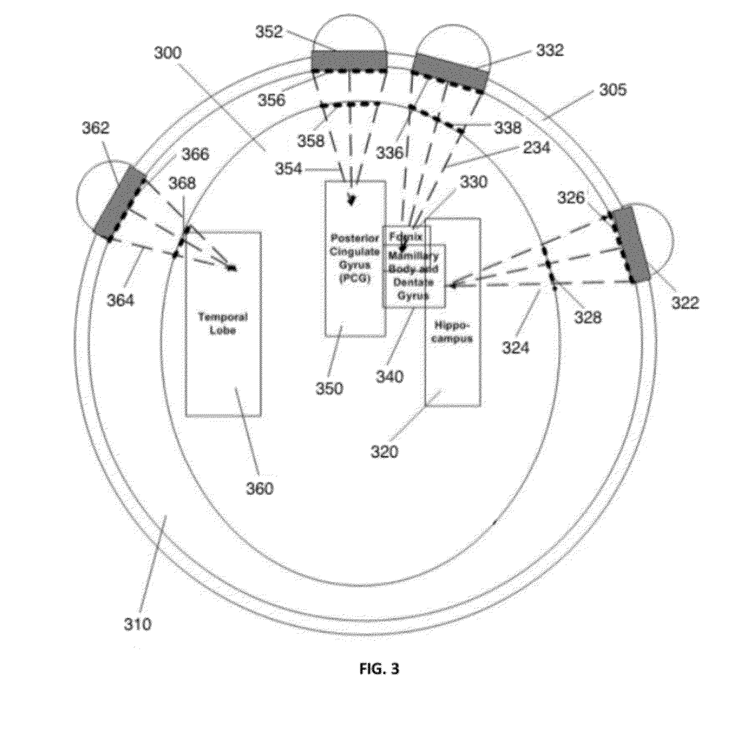

FIG. 3 shows a set of ultrasound transducers targeting to treat Alzheimer's Disease and other dementias. Head 300 contains the five targets, Hippocampus 320, Formix 330, Mamillary Body and Dentate Gyms 340, Posterior Cingulate Gyms (PCG) 350, and Temporal Lobe 360, all of which are to be up regulated. Because of their close proximity the Mamillary Body and Dentate Gyms are together considered a single combined target because they generally would be stimulated together and in terms of ultrasound transducers, Formix 330 would be stimulated generally with the same ultrasound transducers as that for the Mamillary Body and Dentate Gyms. Note that while these five targets are covered here, fewer can work as well, or an addition or substitution of other targets identified in the future. These targets are hit by ultrasound from transducers 322, 332, 352 and 362 fixed to track 305. Ultrasound transducer 322 with its beam 324 is shown targeting Hippocampus 320, transducer 332 with its beam 334 is shown targeting Formix 330 plus Mamillary Body and Dentate Nucleus 340, transducer 352 with its beam 354 is shown targeting the Posterior Cingulate Gyms 350, and transducer 362 with its beam 364 is shown targeting Temporal Lobe 360. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 310 is interposed with one mechanical interface to the frame 305 and ultrasound transducers 322, 332, 352, and 362 (completed by a layer of ultrasound transmission gel layers 326, 336, 356, and 366) and the other mechanical interface to the head 300 (completed by a layers of ultrasound transmission gel 328, 338, 358, and 368). In another embodiment the ultrasound transmission gel is placed around the entire frame 305 and entire head 300.

For any of the cases above, other embodiments have multiple ultrasound transducers with intersecting beams focused on the same target. In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, and medications.

The invention allows stimulation adjustments in variables such as, but not limited to, intensity, firing pattern, frequency, pulse duration, firing pattern, phase/intensity relationships, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of deep-brain neuromodulation using ultrasound stimulation, the method comprising:
aiming one or a plurality of ultrasound transducer at one or a plurality of neural targets, and
applying pulsed power to the ultrasound transducer via a control circuit,
whereby the condition treated is selected from the group consisting of Autism Spectrum Disorders and Alzheimer's Disease or other dementias.

2. The method of claim 1, further comprising aiming an ultrasound transducer neuromodulating motor-disorder-related neural targets in a manner selected from the group of up-regulation, down-regulation.

3. The method of claim 1, wherein the effect is chosen from the group consisting of acute, Long-Term Potentiation, and Long-Term Depression.

4. The method of claim 1, wherein for the treatment of Autism Spectrum Disorders one or a plurality of targets are selected from the group consisting of Anterior Cingulate Gyms, Parietal Lobe, Amygdala, and Caudate Nucleus.

5. The method of claim 1 wherein for the treatment of Alzheimer's Disease and other dementias one or a plurality of targets are selected from the group consisting of Hippocampus, Formix, Mamillary Body and Dentate Gyms, Posterior Cingulate Gyms, and Temporal Lobe.

6. The method of claim 1 wherein a single ultrasonic transducer aimed at a given target is replaced by a plurality of ultrasonic transducers whose beams intersect at that target.

7. The method of claim 1, wherein the acoustic ultrasound frequency is in the range of 0.3 MHz to 0.8 MHz.

8. The method of claim 1, where in the power applied is selected from the group consisting of less than 60 mW/cm² and greater than 60 mW/cm² but less than that causing tissue damage.

9. The method of claim 1 wherein modulation frequency of lower than approximately 500 Hz is divided into pulses 0.1 to 20 msec. repeated at frequencies of 2 Hz or lower for down regulation.

10. The method of claim 1 wherein modulation frequency of approximately 500 Hz or higher is divided into pulses 0.1 to 20 msec. repeated at frequencies higher than 2 Hz for up regulation.

11. The method of claim 1, wherein the focus area of the pulsed ultrasound is 0.5 to 150 mm in diameter.

12. The method of claim 1, wherein mechanical perturbations are applied radially or axially to move the ultrasound transducers.

13. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

14. The method of claim 1, wherein ultrasound therapy is combined with or replaced by one or more therapies selected from the group consisting of Transcranial Magnetic Stimulation (TMS), deep-brain stimulation (DBS), application of optogenetics, radiosurgery, Radio-Frequency (RF) therapy, and medications.
